(19) United States
(12) Patent Application Publication
(10) Pub. No.: US 2017/0028107 A1
(43) Pub. Date: Feb. 2, 2017

(54) KIND OF ABSORBABLE HIGH STRENGTH AND TOUGHNESS CORROSION-RESISTANT ZINC ALLOY IMPLANT MATERIAL FOR HUMAN BODY

(71) Applicant: XI'AN ADVANCED MEDICAL TECHNOLOGY CO., LTD., Xi'an (CN)

(72) Inventors: Gongyao ZHOU, Xi'an (CN); Gongqi QU, Xi'an (CN); Haibo GONG, Xi'an (CN)

(73) Assignee: XI'AN ADVANCED MEDICAL TECHNOLOGY CO., LTD., Xi'an (CN)

(21) Appl. No.: 15/102,355

(22) PCT Filed: Jan. 28, 2014
(86) PCT No.: PCT/CN2014/071611

§ 371 Date: Jun. 7, 2016

(30) Foreign Application Priority Data
Dec. 26, 2013 (CN) 201310756776.1

(51) Int. Cl. A61L 31/02; C22C 18/02; A61L 31/14; A61L 27/50; A61L 27/04; A61L 27/58

(52) U.S. Cl. CPC A61L 31/022; A61L 27/047; A61L 27/58; A61L 31/148; A61L 27/50; A61L 31/14; C22C 18/02; A61L 2430/02

(57) ABSTRACT

A kind of absorbable high strength & toughness corrosion-resistant zinc alloy implant material for human body, the elemental constituent and quality percent: Ce0.001%-2%, Mg0.001%-2%, Ca0.001%-2%, Cu0.01%-3%, others are Zn. Above-mentioned zinc alloy material is prepared as absorbable medical implant by using conventional method in this field, especially for intravascular stent, orthopedic implants (bone nail or plate etc.). Zinc alloy material created by this invention has high corrosion-resistant and high strength & toughness, the medical implant prepared by this can be absorbed by the body without side effects.

### TECHNICAL FIELD

The invention involves in a kind of absorbable high strength & toughness corrosion-resistant zinc alloy implant material for human body, which belongs to medical material field.

### BACKGROUND TECHNOLOGY

Intravascular stent is a implant wire-mesh like instrument that is used to treat vascular disease such as coronary heart disease, with the length of about 8-23 mm, about 2.5-5 mm diameter. At present, the mainstream drug eluting stent (DES) is expandable cobalt-chromium alloy wire mesh, which provides mechanical support for stenosis focus vessel, the drug eluting on the stent can continuously release drug within about one month to inhibit smooth muscle and lymphocyte growth, reduce inflammatory reaction and immunoreaction. For a long period, patients in China remained unaware of stent, because coronary heart disease incidence is was extremely low in China in the past. With rise of national income yearly and improvement of people's living standard and dietary structure, coronary heart disease in recent years in China is on the upward, increasing by 42.2% for male, and 12.5% for female, and tends to younger people. It is reported from foreign medical field that intravascular stent must be installed when coronary heart disease develops a certain level to prevent myocardial infarction. In the 21st century, with the advent of domestic stent, the number of patients for intravascular stent implanting operation has surged, some patients with serious symptoms implant 7-8 stents successively in vessel. A cardiologist from Beijing Fuwai Hospital said, the total number of patients who die from acute coronary annually exceed one million in past several years, if these people install intravascular stent earlier, mortality can reduce by at least 50%, so it is extremely urgent to fully promote and apply intravascular stent.

Traditional intravascular stent is generally made by adopting non-absorbable metal, and the preparation procedure is divided into two steps, first, metal ingot is made into pipe by extrusion molding, then pipe is processed preciously into mesh stent by laser cutting technology. Its disadvantages include that the metal is nondegradable, fails to take out, and is easy to cause late thrombosis if retaining in vessel, in addition, laser cutting technology has low efficiency, high cost. Large number of clinical cases prove that traditional stent is quite effective in reducing stenosis reconstruction rate, but thrombosis rate reaches up to 3%-9% after patients implant this kind of stent for 1-5 years, and post-thrombotic mortality reaches up to 30%. Late thrombosis occurred clinical frequently is exactly technical bottleneck that traditional stent is hard to break though, the root reason is the material that DES used can not be degraded and absorbed by human body, only permanently stayed in vessel of focus. However, vessel only needs 1-3 months of mechanical support to reconstruct vessel function, the vessel has recovered basically after 3 months, and stent in vessel may cause many side effects for patients.

At the same time, bone nail and plate are common medical implant for fixing fracture and ligament injury, especially for fracture is closed to joint and extends to intrajoint, bone nail and plate are used to fix them. Ageing is serious in modern world, osteoporosis patients increase year by year, accidents such as car accidents or falling down often cause comminuted fracture.

Similar problems such as vessel stent also appear in traditional bone nail and plate industry, nondegradable metal is failed to take out, secondary operation must be conducted, which causes enormous wound for patients. Meanwhile, traditional bone nail and plate have high strength, and easy to bring stress shielding, causing injured bone tissue is difficult to regenerate and healing. Traditional polymer bioabsorbable bone bone nail and plate have low strength, and fracture accident often occur during clinical application.

Because there are many problems for traditional medical implant materials and body absorbable medical materials, especially for intravascular stent, orthopaedic implants, it is the focus of research and development of above-mentioned fields. Zinc-alloy is the most extensive discussion in recent years, the material can be fully absorbed by organism, and the strength and toughness are higher than polymer, elasticity modulus is more similar to human bone. But zinc-alloy has very poor corrosion resistance, and it is degraded and absorbed quickly in the human body, failed to provide enough mechanical support time.

### INVENTION CONTENT

The invention mainly aims to invent a absorbable and degradative zinc-alloy implant material by human body, and prepare absorbable medical implant by using this material, especially intravascular stent, bone nail or plate. Zinc-alloy implant material with anti-corrosion, high strength and toughness can realize three technical indicators: fully absorbed, proper mechanical strength and enough mechanical support time. Absorbable stent may make vessel recover when treating coronary heart disease, which is impossible to traditional metal stent. Because there is no permanent metal left in the body, the vessel for patients treated by absorbable stent may be able to move, stretch and pulse, liked vessel never be treated. Absorbable bone bone nail and plate enable to match regenerative healing of bone tissue, its mechanical strength matched to bone tissue, and neither cause stress shielding, nor facture accident.

Absorbable high strength and toughness corrosion-resistant zinc alloy implant material by human body stated in technical scheme of the invention, elemental constituent and quality percent: Ce0.001%˜2%, Mg0.001%˜2%, Ca0.001%˜2%, Cu0.01%˜3%, others are Zn.

Based on composition of above alloy material, and in order to mechanical performance and biological corrosion resistance, mass ratio composition optimized for zinc-alloy implant material: Ce0.001%˜1.5%, Mg0.002˜1.5%, Ca0.001˜1.5%, Cu0.005%˜2%, others are Zn.

In order to obtain the best mechanical property and biological corrosion resistance, the invention strictly controls the content of impurities such as Al and Mn: Zn purity is greater than or equal to 99.999%; Ce purity is greater than or equal to 99.99%; Mg purity is greater than or equal to 99.99%; Ca purity is greater than or equal to 99.99%; Cu purity is greater than or equal to 99.99%; total impurities is less than 0.1% except Zn, Ce, Mg, Ca and Cu.

Above-mentioned alloy material prepared for this invention is prepared absorbable medical implant by conventional method in the field, and absorbable medical implant is selected for intravascular stent or orthopedic implants. Orthopedic implants can be either bone nail or plate.

We made a lot of experiments research for zinc-alloy material prepared in examples 1-3 below, the main result is as follows:

Simulated body fluid immersion test proved that, zinc-alloy surface formed low dissolved protection layer, therefore, degradation speed is much lower than magnesium alloy, only 0.15 mm/year, which can guaranteed that intravascular stent provides radial support with vessel focus for more than 6 months, mechanical support duration for fixation and bone plate reaches at least half a year. Slow and uniform degradation process allows vessel to generate gradually, and recover movement, expansion and pulsation as same as health vessel.

Tensile test result suggested that zinc-alloy elasticity modulus was about 80 GPa, yield strength can reached up to 230 NPa, with excellent mechanical performance. The research found that elasticity modulus of human cortical bone is 3˜20 GPa, yield strength is 35˜280 GPa, alloy prepared in this invention matched well with mechanical property of human cortical bone.

Therefore, beneficial effects of absorbable zinc-alloy material created by this invention can be summarized as:

can be absorbed by human body to avoid pain caused by secondary operation;

corrosion resistance is higher than magnesium alloy, and degradation velocity is reduced substantially, longer mechanical support can be provided to avoid, intravascular stent and bone nail implants fail early;

mechanical strength is higher than polymer, high toughness, easy to manufacture, elasticity modulus matches well with human body;

realize high corrosion resistance and toughness.

### SPECIFIC EXAMPLES

The following is specific examples of this invention, which are used to state technical scheme of problems in application document and help technicians understand the invention, but the implementation of technical scheme is not limited to these examples.

### Example 1

A kind of absorbable zinc-alloy implant material by human body, and elemental constituent and quality percent is: Ce0.1%, Mg0.5%, Ca0.1%, Cu1.5%, others are Zn.

Above components are prepared as zinc-alloy material finally according to common smelting and processing process in the field.

### Example 2

A kind of absorbable zinc-alloy implant material by human body, and elemental constituent and quality percent is: Ce0.001%, Mg1.5%, Ca0.001%, Cu0.01%, others are Zn.

### Example 3

A kind of absorbable zinc-alloy implant material by human body, and elemental constituent and quality percent is: Ce1.5%, Mg0.001%, Ca1.5%, Cu2%, others are Zn.

Components for above zinc-alloy material in example 1-3 are prepared as zinc-alloy material finally according to common smelting and processing process in the field; then prepared as absorbable medical implant by these material and conventional method.

1-7. (canceled)

8. An absorbable high strength and toughness corrosion-resistant zinc alloy implant material for human body, wherein an elemental constituent and percent by mass of the elemental constituent in the zinc alloy implant material are as follows:
Ce in a range of from 0.001% to 2%,
Mg in a range of from 0.001% to 2%,
Ca in a range of from 0.001% to 2%
Cu in a range of from 0.01% to 3% and
Zn is the remaining content.

9. The zinc alloy implant material according to claim 8, wherein
the percent by mass Ce is in a range of from 0.001% to 1.5%,
the percent by mass Mg is in a range of from 0.002% to 1.5%,
the percent by mass Ca is in a range of from 0.001% to 1.5%,
the percent by mass Cu is in a range of from 0.05% to 2%, and
Zn is the remaining content.

10. The zinc alloy implant material according to claim 9, wherein
the percent by mass Ce is 0.1%,
the percent by mass Mg is 0.5%,
the percent by mass Ca is 0.1%, and
the percent by mass Cu is 1.5%.

11. The zinc alloy implant material according to claim 8, wherein a purity of the elemental constituents used to form the zinc alloy implant material is as follows:
Zn purity is greater than or equal to 99.999%,
Ce purity is greater than or equal to 99.99%,
Mg purity is greater than equal to 99.99%,
Ca purity is greater than or equal to 99.99%, and
Cu purity is greater than or equal to 99.99%.

12. The zinc alloy implant material according to claim 8, wherein total inclusion in the zinc alloy implant material is less than 0.1% except Zn, Ce, Mg, Ca and Cu.

13. The zinc alloy implant material according to claim 12, wherein zinc allay implant material is in the shape of an absorbable medical implant, wherein the absorbable medical implant is selected from the group consisting, of an intravascular stent and orthopedic implants.

14. The zinc alloy implant material stated in claim 8, wherein the zinc allay implant material is in the shape of an orthopedic implant, wherein orthopedic implant is a bone nail or plate.
